(19) United States
(12) Patent Application Publication
(10) Pub. No.: US 2023/0301641 A1
(43) Pub. Date: Sep. 28, 2023

(54) Endoscopic Vessel Harvesting System Including a Visual Indicator

(71) Applicants: Robert Rodela, Corpus Christi, TX (US); Luis Reyes, McAllen, TX (US)

(72) Inventors: Robert Rodela, Corpus Christi, TX (US); Luis Reyes, McAllen, TX (US)

(21) Appl. No.: 18/126,973

(22) Filed: Mar. 27, 2023

Related U.S. Application Data
(60) Provisional application No. 63/323,612, filed on Mar. 25, 2022.

(51) Int. Cl. A61B 17/00; A61B 17/02

(52) U.S. Cl. CPC A61B 17/00008; A61B 17/0218

(57) ABSTRACT

An endoscopic vein harvesting system may include an endoscope including a proximal end having one or more of a collar or a handle and including a distal end. The system may include a component formed from a transparent material and configured to couple to the distal end of the endoscope. The system may include one or more visual indicators formed on or within a selected portion of the component. In some implementations, the one or more visual indicators may assist a harvester to determine a position of the component relative to a vein to be harvested.

### CROSS-REFERENCE TO RELATED APPLICATION(S)

This application is a non-provisional of and claims priority to U.S. Provisional Patent Application No. 63/323,612 filed on Mar. 25, 2022 and entitled “Endoscopic Vessel Harvesting System Including a Visual Indicator”, which is incorporated herein by reference in its entirety.

### FIELD

The present disclosure is generally related to surgical devices and methods for dissection and removal of blood vessels from a patient's body and, more particularly, to endoscopic vessel harvesting systems, components, and methods.

### BACKGROUND

The phrase “heart disease” refers to several types of hear conditions. The most common type of heart disease in the United States is coronary artery disease, which may affect blood flow to the heart. Coronary artery disease may include narrowing, blocked, or partially blocked veins or arteries in a patient's heart, which may restrict blood flow, and which may result in a heart attack or cardiac arrest. Sometimes, treatments for heart disease may include using angioplasty (which includes temporarily inserting a device into the affected area of the artery and inflating a tiny balloon to widen the area) or inserting a stent (which includes permanently inserting a small wire mesh tube into the affected area of the artery to hold the artery open. If the insertion of the stent is not successful or if the insertion was successful but the artery has narrowed again (such as by plaque buildup), treatment of the heart disease may require coronary bypass surgery.

Coronary bypass surgery is a well-known technique for improving blood flow in and around a patient's heart. Coronary bypass surgery involves redirecting blood flow around a section of the blocked artery in the patient's heart by taking a healthy blood vessel from the patient's leg (saphenous vein), arm (radial artery), or chest (internal mammary artery) and connecting it to the blocked artery on both sides of the blockage to provide a new pathway for blood flow to the heart muscle, bypassing the blockage.

In the past, harvesters often used an open technique for vein harvesting in which a long incision was made to expose the vein to allow the harvester to extract it. However, such incisions carried a relatively high incidence of wound complications and pain.

For at least the last decade, endoscopic vein harvesting has been the method of choice to harvest the greater saphenous vein, which is the most widely used conduit in coronary bypass surgery. The saphenous vein is often used because of the relative ease of harvesting and the length of the vein, which may allow for multiple bypass segments. Endoscopic vein harvesting may be performed by making a small incision near an end of the vein to be extracted and by inserting an endoscope into the incision. The endoscope includes a long, thin tube that may include a camera or optical sensor configured to capture visual data including images of the end of the endoscope, allowing the harvester to see inside the patient's body. Typically, the end of the endoscope includes a cap or dissection tip that may be used by the harvester to carefully separate the vein from the surrounding tissue, and various accessories may be used to cauterize branches and to cauterize, tie, or otherwise ligate the vessel at a proximal site and a distal site. One or more of the accessories may then be used to transect the vessel at the proximal and distal sites before removing the selected vein from the body for use in the bypass surgery.

### SUMMARY

Anatomical vein harvesting procedures are becoming increasingly common for treating heart disease. Such procedures often use an endoscope that may fit through a small incision at one end of the vein and that may be used to separate the vein from surrounding tissue and to extract the vein through the small incision. The endoscope may include a clear dissection tip or cap through which optical sensors may capture video of the surrounding tissue and the vein of interest, and the harvester performing the procedure may rely on the video data to determine his or her actions during the procedure. However, to enhance visibility for the harvester, many of the components (such as the dissection tip, manipulating devices, and even the edges of a bisector device) may be clear. During the procedure, such clear components may be difficult to see against the surrounding tissue within the video data, and the harvester may struggle to discern the precise position of the component relative to the vein.

Embodiments of components used during an endoscopic vein harvesting procedure are disclosed herein that may include a visual indicator formed on one or more of a dissection tip, a bisector, or a manipulation device, such as a cradle or holder. The visual indicator may include a mark or shape formed on a tip or edge of the component. The visual indicator may be formed have a color that stands out visually from (has a high contrast relative to coloration of) the surrounding biological material. In an example, the visual indicator may be formed from a fluorescent green color, and the harvester (doctor or user) may be able to discern the visual indicator within the visual data presented on a display so that the harvester can visually determine the precise position of the component (or the distal tip of the endoscope) relative to the vein to be harvested.

In some implementation, a device may include a first portion configured to couple to a distal end of an endoscope and a second portion configured to engage biological tissue of a patient. The second portion may include one or more visual indicators. The visual indicator may include a dot, crosshairs, an arrow, a triangle, or another visible feature or shape formed on or within the second portion. The visual indicator may be formed from a fluorescent color of ink that has a high contrast relative to the colors presented by the biological material so that the second portion may be readily determined within the optical data presented on a display that is coupled to the endoscope. The visual indicator may provide a visual alignment feature to assist the harvester to precisely align the component to the selected vein.

In some implementations, an endoscopic vein harvesting system may include an endoscope including a proximal end having one or more of a collar or a handle and including a distal end. The system may include a component formed from a transparent material and configured to couple to the distal end of the endoscope. The system may include one or more visual indicators formed on or within a selected portion of the component. In some implementations, the one or more visual indicators may assist a harvester to determine a position of the component relative to a vein to be harvested.

In other implementations, an endoscopic vein harvesting system may include an endoscope including a proximal end including one or more of a collar or a handle and including a distal end to be inserted through an incision into a patient. The system may include a component formed from a transparent material and configured to couple to the distal end of the endoscope prior to insertion. The component may include one or more visual indicators formed on or within a selected portion of the component to facilitate positioning of the component relative to a vein to be harvested. The system may include a computing system coupled to the endoscope and configured to receive optical data corresponding to a view through the distal end of the endoscope. The optical data may include images of the one or more visual indicators. The computing system may include a display and may be configured to present at least a portion of the optical data including the one or more visual indicators to the display.

### BRIEF DESCRIPTION OF THE DRAWINGS

The detailed description is set forth with reference to the accompanying figures. In the figures, the left most digit(s) of a reference number identifies the figure in which the reference number first appears. The use of the same reference numbers in different figures indicates similar or identical items or features.

FIG. 1A depicts a view of a portion of a patient's leg including a portion of an endoscopic system including an endoscope with a dissection tip that includes a visual indicator and that is inserted in a leg for harvesting of a saphenous vein, in accordance with certain embodiments of the present disclosure.

FIG. 1B depicts a view of the dissection tip of the endoscope of FIG. 1A including the visual indicator, in accordance with certain embodiments of the present disclosure.

FIG. 1C depicts an image captured by optical sensors of the endoscope of the dissection tip in situ and including a visual indicator, in accordance with certain embodiments of the present disclosure.

FIG. 2 depicts an alternative embodiment of an endoscope including a manipulating device and a bisector device, in accordance with certain embodiments of the present disclosure.

FIG. 3A depicts an image of an implementation of a manipulator device for holding a vein to be harvested and including a visual indicator, in accordance with certain embodiments of the present disclosure.

FIG. 3B depicts an image of an implementation of a manipulator device for holding a vein to be harvested and including a visual indicator, in accordance with certain embodiments of the present disclosure.

FIG. 4 depicts an image of an endoscopic vein harvesting procedure including an endoscope with an implementation of a dissection cap for separating the vein to be harvested from surrounding biological material and including a visual indicator, in accordance with certain embodiments of the present disclosure.

FIG. 5 depicts a block diagram of a system including an endoscopic vein harvesting system including any of the devices of FIGS. 1A-4, in accordance with certain embodiments of the present disclosure.

While implementations are described in this disclosure by way of example, those skilled in the art will recognize that the implementations are not limited to the examples or figures described. The figures and detailed description thereto are not intended to limit implementations to the particular form disclosed, but on the contrary, the intention is to cover all modifications, equivalents, and alternatives falling within the spirit and scope as defined by the appended claims. The headings used in this disclosure are for organizational purposes only and are not meant to limit the scope of the description or the claims. As used throughout this application, the word “may” is used in a permissive sense (in other words, the term “may” is intended to mean “having the potential to”) instead of in a mandatory sense (as in “must”). Similarly, the terms “include”, “including”, and “includes” mean “including, but not limited to”.

### DETAILED DESCRIPTION OF ILLUSTRATIVE EMBODIMENTS

Endoscopic vein harvesting procedures have become increasingly common for bypass surgeries for treating blood flow problems within and around the heart. Embodiments of systems, methods, and devices described below include components for use with an endoscope, such as a dissection tip, a bisector, a manipulation device, or other devices. Each of the components may include a visual indicator that provides a visible guide to facilitate the orientation and manipulation of the component relative to the vein during the harvesting procedure. The visual indicator may have a selected shape, such as a line, crosshairs, and arrow, a dot, a triangle, or another shape that may be used by the harvester to align or position the component relative to the vein. Additionally, the visual indicator may be formed from a selected color that has a high contrast relative to the biological tissue, such as fluorescent green or another color, so that the visual indicator can be seen in the video data on a computer monitor associated with the endoscopic vein harvesting system.

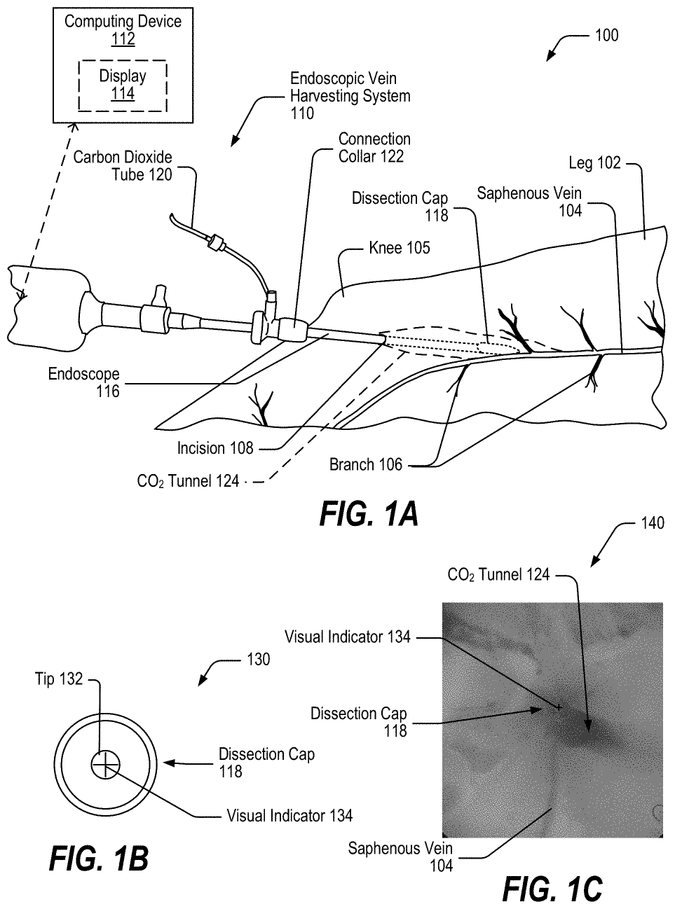

In some implementations, a system may include an endoscopic vein harvesting device coupled to a computing system that includes a display. The endoscopic vein harvesting system may include an endoscope including a proximal end coupled to the computing system and a distal end coupled to one or more components configured to facilitate extraction of a selected vein. The endoscope may include one or more optical sensors configured to capture optical data associated with the distal end of the endoscope and to provide the optical data to the computing system for presentation on the display. At least one of the one or more components may include a visual indicator having a color that has a high contrast relative to the biological material of the patient, and the optical data may include the visual indicator. In some implementations, the component may be formed from a substantially transparent material, which may provide little to no contrast relative to the biological material during the procedure. The visual indicator may render at least a portion of the component visible to the harvester to facilitate positioning of the distal end of the endoscope. An example of the endoscopic vein harvesting system is described below with respect to FIG. 1A.

FIG. 1A depicts a view 100 of a portion of a patient's leg 102 including a portion of an endoscopic system including an endoscope 116 with a dissection tip 118 that includes a visual indicator and that is inserted in the leg 102 for harvesting of a saphenous vein 104, in accordance with certain embodiments of the present disclosure. In this example, the portion of the leg 102 includes the knee 105 and portions of the leg on both sides of the knee 105. The leg 102 includes a saphenous vein 104 that extends along the length of the leg 102. Typically, the saphenous vein 104 may include branches 106 that extend away from the saphenous vein 104 and that facilitate blood flow to the muscle and tissue of the leg 102. To harvest a segment of the saphenous vein 104 for reuse in a bypass surgery, the saphenous vein 104 is separated from the surrounding biological material, the branches 106 are transected, and the transected ends of the branches are cauterized to seal the cuts.

The endoscope 116 may include a thin, flexible tube with a light and a camera near a distal end, which may be inserted through an incision 108 and pushed into the patient's leg 102. During the insertion process and during the harvesting procedure, a camera associated with the distal end of the endoscope 106 may capture optical data that may be presented on a display 114 of a computing device 112. The harvester (a doctor using the endoscope 116) may view the display 114 while moving or otherwise manipulating the endoscope 116 to view inside of the patient and to harvest the saphenous vein 104.

The endoscopic vein harvesting system 110 may include one or more components coupled to a distal end of the endoscope 116. In this example, a dissection tip or cap 118 is coupled to the distal end. In other implementations, a component, such as a bisector, a manipulation device, a dissection cap 118 (sometimes referred to as a “dissection tip”), or another component may be coupled to the distal end of the endoscope 118. The endoscope vein harvesting system 110 may include a connection collar 122 that may be coupled to a carbon dioxide tube 120 or to other tubes to receive a fluid. Additionally, the endoscope 116 may be coupled to a handle and to other connection collars, which may facilitate insertion of additional components, such as a cauterizer, a bisector, and so on.

In this example, the dissection cap or tip 118 may be formed from a clear material that is substantially transparent, allowing the optical sensors (cameras) to capture images through the distal end of the endoscope 116. The harvester may insert the endoscope 116 with the dissection cap or tip 118 through the incision 108 and may use the dissection cap or tip 118 to separate the saphenous vein 104 from surrounding biological material. In some implementations, the harvester may inject carbon dioxide under pressure via the carbon dioxide tube 120 to facilitate separation of the tissue to form a carbon dioxide tunnel 124.

In an example, a harvester (the doctor) may create an incision 108 over the saphenous vein 104. The harvester may attach the dissection cap or tip 118 to the distal end of the endoscope 116 and may insert the endoscope 116 into the incision 108. Optical data from the sensors of the endoscope 116 may be presented on a display 114 of the computing device 112 to allow the harvester to visualize the vein 104 and the surrounding tissue on the display 114. The harvester may then insufflate carbon dioxide gas through the endoscopic vein harvesting system 110 via the carbon dioxide tube 120 to create a carbon dioxide tunnel 124. The harvester may advance the endoscope 116 to use the dissection cap or tip 118 to perform anterior and posterior dissection of the saphenous vein 104 from the surrounding biological material.

When the endoscope 116 encounters a branch 106 extending from the saphenous vein 104, the harvester may use one or more of a vein manipulation device or a bisector to seal and transect the branches 106 one at a time. The harvester may utilize the manipulation device to manipulate the saphenous vein 104 to determine that the section of the saphenous vein is free and that no branches remain intact. The harvester may then clamp the saphenous vein 104, transect and ligate both ends of the saphenous vein (the end to be harvested that is furthest from the incision 108), and then remove the selected portion of the saphenous vein 104 through the incision 108. The harvested portion may then be used for bypass surgery, for example.

In some implementations, the dissection cap or tip 118 or other vein manipulation components may be difficult to see in the image data relative to the surrounding tissue. In particular, when the dissection cap or tip 118 or the other vein manipulation components are made from clear or transparent materials to facilitate imaging, position of the dissection cap or tip 118 or the other vein manipulation components relative to the saphenous vein 104 may be difficult if not impossible to discern from the optical data. Accordingly, the dissection cap or tip 118 or other vein manipulation components may include a visual indicator having a color configured to contrast with the color of surrounding tissue to enable the harvester to determine the precise position of the dissection cap or tip 118 or the other vein manipulation components in situ from the optical data on the display 114. An example of a dissection cap or tip 118 or the other vein manipulation component that include a visual indicator is described below with respect to FIG. 1B.

FIG. 1B depicts a view 130 of the dissection cap 118 of the endoscope 116 of FIG. 1A including the visual indicator 134, in accordance with certain embodiments of the present disclosure. In this example, the view 130 shows the dissection cap 118 from the perspective of the optical sensors within the endoscope 116. The dissection cap 118 may be formed from a clear material, such as an antimicrobial plastic, glass, or other material, which may be transparent to allow the sensors to capture optical data through the dissection cap 116, providing little or no visible contrast relative to the surrounding biological material.

In this example, the dissection cap 118 has an elongate shape with a curved tip 132, which may be used by the harvester to separate the vein from the surrounding biological material. To assist the harvester so that he or she can readily determine the position of the tip 132 in situ, the tip 132 may include a visual marker 134. In this example, the visual marker 134 is depicted as a crosshair, but other shapes are also possible, such as a dot, a triangle, a square, a circle, or another shape. The visual marker 134 may make it possible to determine the position of the tip 132 against the background of the biological material. The visual marker 134 may be printed on or embedded within the tip 132 and may be selected to have a color that provides a visible contrast between the visual marker 134 and the biological material.

In an example, the surrounding biological material may include white, beige, red, and other colors that are common to biological tissue, such as dermal layers, veins, fascia, and other biological material that may be commonly encountered in vein harvesting procedures. The visual marker 134 may be implemented in a neon green color or another color that is not typically encountered in an operating environment. An example of a dissection cap or tip 118 with a visual indicator 134, in situ, is described below with respect to FIG. 1C.

FIG. 1C depicts an image 140 of the dissection cap 118 in situ and including a visual indicator 134 captured by optical sensors of the endoscope 116, in accordance with certain embodiments of the present disclosure. The image 140 may be an example of the optical data presented on the display 114 of the computing device 112. The image 140 shows a portion of the carbon dioxide tunnel 124 and the saphenous vein 104. The dissection cap 118 is almost invisible against the background of the biological material, but the visual indicator 134 can be seen against the background, enabling the harvester to determine the precise location of the tip 132.

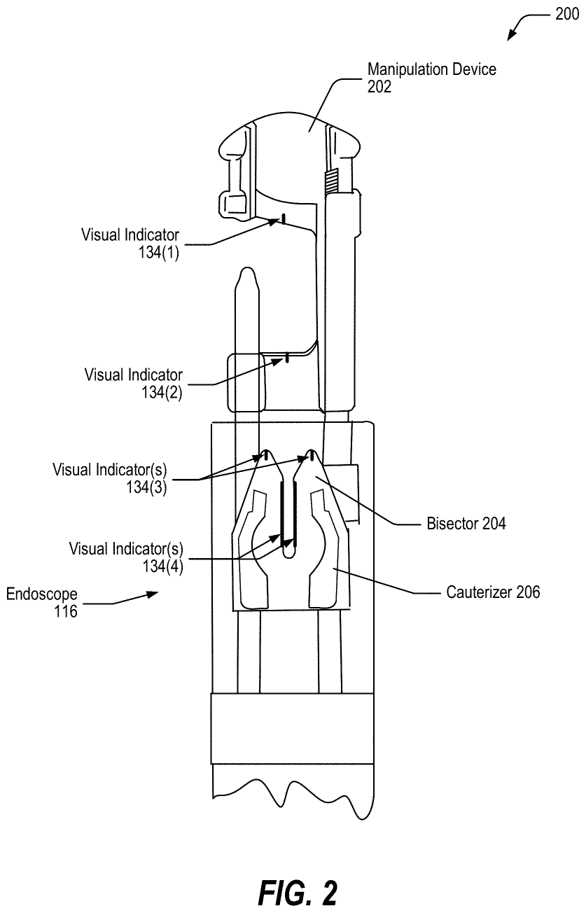

It should be appreciated that the dissection cap or tip 118 is one possible example of an endoscopic component that can be implemented with a visual indicator 134. One or more of the size, shape, or color of the visual indicator 134 may be selected to provide a visible marker 134 that may be used by the harvester (the doctor) to determine the position of the dissection cap or tip 118 relative to the vein 104 to be harvested. Additionally, it should be understood that the visual indicator 134 may also be applied to other components that may be coupled to or integrated with the distal end of the endoscope 116. An example of a manipulation device with a visual indicator that is coupled to the distal end of the endoscope 116 is described below with respect to FIG. 2.

FIG. 2 depicts a view 200 of an alternative embodiment of an endoscope 116 including a manipulating device 202 and a bisector device 204, in accordance with certain embodiments of the present disclosure. In this example, the endoscope 116 may include a manipulating device 202, which may be curved or flat and which may be configured to fit under or partially around the vein 104 so that the harvester can manipulate and position the vein 104. In some implementations, the manipulation device 202 may include one or more visual indicators 134(1) and 134(2) that may be used by the harvester to center the vein onto the manipulation device 202. In this example, single visual indicators 134(1) and 134(2) are provided in alignment on both ends of the manipulation device 202 with a gap in between. In other implementations, a pair of visual indicators 134(1) and 134(2) may be provided on both ends of the manipulation device 202. Other configurations are also possible.

The endoscope 116 may include a bisector 204, which may include a pair of visual indicators 134(3) on opposing sides to provide visual data on the display 114 that may be used by the harvester (the doctor) to center the branches 106 between the visual indicators 134(3). In some implementations, in addition to or in lieu of the visual indicators 134(3), the bisector 204 may include visual indicators 134(4) along interior cutting surfaces. The visual indicators 134(3) and 134(4) may enable to harvester to see the precise location of the bisector 204 on the display 114. In some implementations, the bisector 204 may also include a cauterizer 206 to cauterize and seal the branches 106 as the bisector 204 severs them from the vein 104.

In this example, the bisector 204 and the manipulation device 202 may be formed from a transparent material, which may allow the endoscope 116 to capture optical data through them. However, the visual indicators 134 may make the position of the otherwise clear components visible to the harvester to facilitate positioning of the various components relative to the vein 104.

It should be understood that the endoscope 116 and its components (e.g., the bisecting cap 118, the bisector 204, and the manipulation device 202) may vary in size and form depending on the manufacturer. Each of the components may include a visual indicator 134 that may have a selected shape and a color that stands out against the background of the biological material.

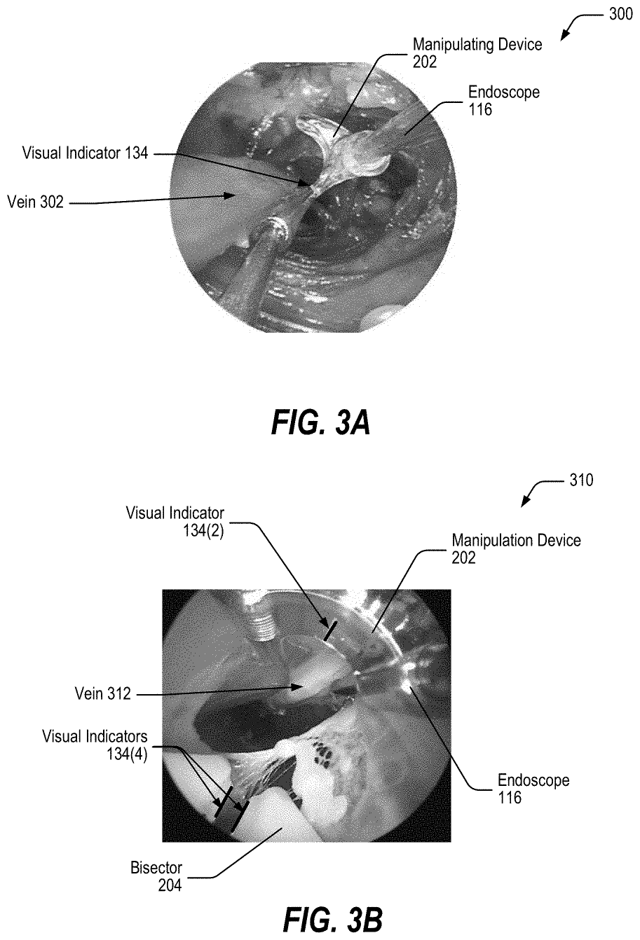

Depending on the implementation, the optical data may be presented in a square, rectangular, or circular view on the display 114. An example of the image data presented in a circular view is depicted in FIG. 3A.

FIG. 3A depicts an image 300 of an implementation, in situ, of a manipulator device 202 for holding a vein 302 to be harvested and including one or more visual indicators 134, in accordance with certain embodiments of the present disclosure. The image 300 may be an example of the optical data that may be presented on the display 114 of the computing device 112 in FIG. 1. In this example, the optical data is presented in a substantially circular view.

The vein 302 may be the saphenous vein 104 in FIG. 1 or may be another vein to be harvested. In the illustrated example, the visual indicator 134 may be presented at a midpoint of the manipulator device 202. The visual indicator 134 is shown as a line extending perpendicular to a vein-contact edge of the manipulator device 202. The visual indicator 134 may have one or more of a size, a color, or a shape configured to assist the harvester (the doctor) to see the edge of the manipulator device 202 and to position the manipulator device 202 relative to the vein 104. The manipulator device 202 of this example has a curved shape to facilitate capture and manipulation of the vein 302.

In other examples, two or three visual indicators 134 may be disposed along the manipulator device 202 to present a visual guide for positioning and alignment. In other implementations, the visual indicators 134 may be presented in different shapes or forms, such as lines, triangles, dots, circles, arrows, and so on. The visual indicators 134 may be formed on the surface or embedded within the manipulator device 202 and may be formed from a selected color that provides a high contrast relative to the background (the biological material). In this context, the background may be substantially red, and the visual indicator 134 may be formed from fluorescent green or another color that has a high contrast relative to the background.

The background colors may vary depending on the tissue and the position of the endoscope 116. In some instances, the background colors may be varied. In other instances, the background colors may be substantially monochrome. In the example, of FIG. 3B, the background biological material includes different shades of the same color (in the original picture, red). In other instances, the background color may be more beige, tan, or brown. An example of an endoscope 116 in situ is described below with respect to FIG. 3B.

FIG. 3B depicts an image 310 of an implementation of a manipulator device 202 for holding a vein 312 to be harvested and including a visual indicator, in accordance with certain embodiments of the present disclosure. The image 310 may be an example of the optical data presented on the display 114 of the computing device 112 of FIG. 1. In this example, the image 310 may be presented as a rectangular shape on the display 114. The vein 312 may be the saphenous vein 104 in FIG. 1 or another vein to be harvested.

In this example, the manipulation device 202 may have a curved shape and may be used to captured and manipulate the vein 312 for harvesting. The manipulation device 202 includes a visual indicator 134(2) at approximately a center point along the curvature. In some implementations, one or more visual indicators 134(2) may be provided on the manipulation device 202, providing a visual guide for positioning and alignment of the manipulation device 202 relative to the vein 312 to be harvested. The image 310 also includes a portion of the bisector 204 including visual indicators 134(4) along opposing cutting edges to provide a visible guide that may be used by the harvester to align the bisector 204 to the branch 106 to be cut.

The inclusion of visual indicators 134 on the various components used in the vein harvesting process provides a number of advantages over conventional components. First, the visual indicator 134 provides a means by which the harvester can see the position of the tip 132 of a dissection cap 118 on the distal end of the endoscope 116 within the view presented on the display 114 of the computing device 112. Unlike conventional components that are difficult, if not impossible, to see in the images presented to the display 114, the visual indicator 134 provides a high contrast marker on the component that can be used for positioning and alignment and that can be readily viewed within the images presented on the display 114. By visually determining the position of the tip 132 from the visual indicator 134, the harvester can readily steer and position the dissection cap 118 to separate the vein 104 from surrounding biological material without inadvertently tearing the vein 104.

When one or more visual indicators 134 are presented on the tips of the bisector 204, the harvester can see the position of the opening of the bisector 204 to facilitate positioning for transecting and cauterizing branches 106. By applying visual indicators 134 to the manipulation device 202, the harvester can better see the position and alignment of the manipulation device 202 relative to the vein 104.

The color, shape, size, position, and number of visual indicators 134 on each component may be selected based on the surgical environment, the procedure to be performed, and so on. In some implementations, the color may be selected based on the background color presented by the biological material so that the visual indicator 134 is clearly visible relative to the background colors and shadows.

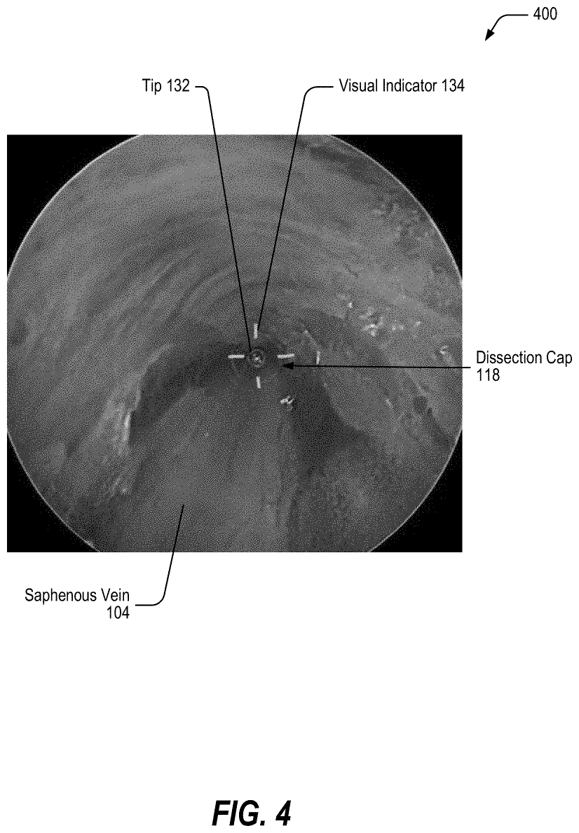

FIG. 4 depicts an image 400 of an endoscopic vein harvesting procedure including an endoscope 116 with an implementation of a dissection cap 118 for separating the vein to be harvested from surrounding biological material and including a visual indicator 134, in accordance with certain embodiments of the present disclosure. In this example, the visual indicator 134 is presented as a set of lines arranged as a sighting reticle surrounding the tip 132 of the dissection cap 118.

In this example, the bisecting tip or cap 118 is transparent, making it difficult to see against the background material. The visual indicator 134 may include lines (as shown), arrows, triangles, or other shapes that surround the tip 132 to facilitate alignment and positioning of the bisecting tip or cap 118. The visual indicator 134 may be formed from a color that has a high contrast relative to the colors presented by the biological material, making the visual indicator 134 easy to see within the optical data presented on the display 114.

The image 400 depicted in FIG. 4 represents another example of a visual indicator 134. Instead of presenting the visual indicator 134 as crosshairs within the tip 132, the visual indicator 134 is not confined to the tip 132. Each of the lines of the visual indicator 134 point toward the tip 132. Other implementations are also possible.

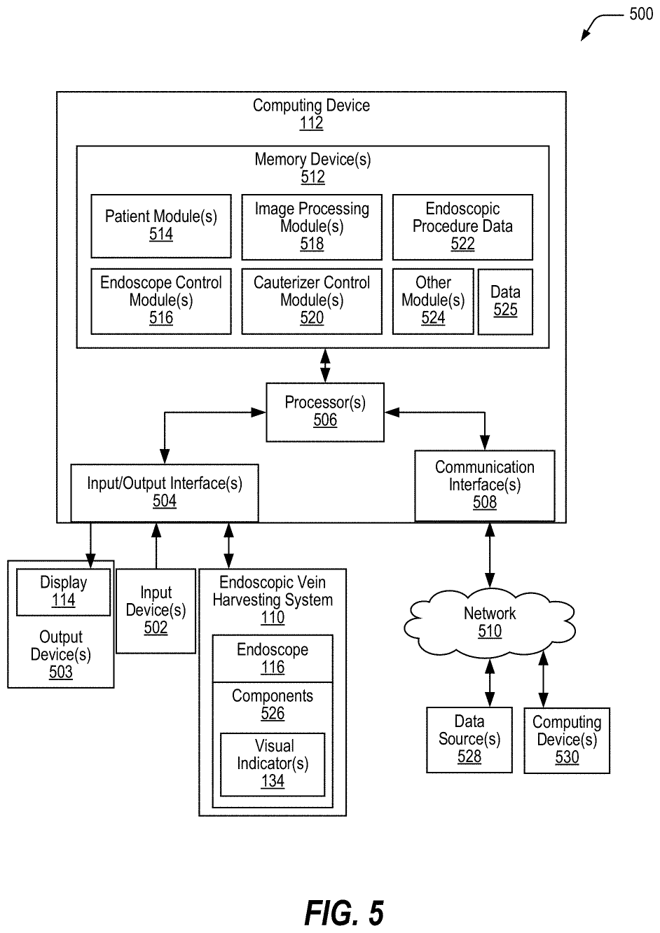

In some implementations, the endoscope 116 may be part of a larger system including a computing device and various components. An example of such a system is described below with respect to FIG. 5.

FIG. 5 depicts a block diagram of a system 500 including any of the endoscopic vein harvesting systems 110 and components described above with respect to FIGS. 1A-4, in accordance with certain embodiments of the present disclosure. The system 500 may include a computing device 112 coupled to a display 114 and to the endoscopic vein harvesting system 110, which may include an endoscope 116 and one or more components 526 (such as the dissection cap 118, the manipulation device 202, or the bisector 204) with visual indicators 134.

The computing device 112 may include one or more input/output (I/O) interfaces 504, which may be coupled to one or more output devices 503 (such as the display 114), one or more input devices 502, and the endoscopic vein harvesting system 110. The one or more output devices 503 may include a speaker, a printer, lights, the display 114, other output devices, or any combination thereof. The one or more input devices 502 may include a keyboard, a microphone, a pointer device (such as a mouse, a trackpad, a stylus, a roller device, or any combination thereof), cameras, scanners, sensors, other input devices, or any combination thereof. In some implementations, one or more of the input devices 502 and the display 114

The computing device 112 may also include one or more communication interfaces 508, which may be configured to communicate data to and to receive data from a network 510. The network 510 may be a proprietary or private communications network, such as a local area network within a hospital. The computing device 112 may communicate with one or more data sources 528 and optionally with one or more other computing devices 530 through the network 510. The computing device 112 may be a standalone computer, such as a desktop computing system, a computer server, or another standalone computing device, which may be integrated into a hospital computing network. The computing devices 530 may include smartphones, tablet computer, laptop computer, other computing devices, or any combination thereof.

The computing device 112 may include one or more processors 506 configured to execute processor-readable instructions. The one or more processors 506 may be coupled to the one or more I/O interfaces 504 and to the one or more communication interfaces 508. The one or more processors 506 may include general purpose processors, digital signal processors, image processors, other processors, or any combination thereof.

The computing device 112 may include one or more memory devices 512 that may be coupled to the one or more processors 506. The memory devices 512 may include one or more of a hard disc drive, a solid-state drive, a flash memory, other non-volatile memory devices, or any combination thereof. The one or more memory devices 512 may be configured to store processor-readable instructions and data.

The memory device 512 may include one or more patient modules 514 that, when executed, may cause the processor 506 to determine and retrieve patient data corresponding to the patient undergoing the vein harvesting procedure from one or more data sources 528 via the network 510. The one or more data sources 528 may include a patient database or other hospital systems.

The memory 512 may include one or more endoscope control modules 516 that, when executed, may cause the processor 506 to communicate with the endoscopic vein harvesting system 110 via the I/O interfaces 504 to receive optical data and other data and optionally to control operation of one or more components. In some implementations, the endoscope control modules 516 may also be used to interact with various functional elements of the endoscopic vein harvesting system 110, such as a carbon dioxide inlet that may be coupled to the carbon dioxide tube 120 (in FIG. 1), or other elements. The endoscope control modules 516 may be configured to send control signals to those components to activate, deactivate, or otherwise control their operation. In some implementations, in addition to or in lieu of control by the endoscope control modules 516, the components may be controlled locally by the harvester by accessing one or more user-selectable control elements (switches, buttons, knobs, or other control elements) on a handle of the endoscope 116 by the harvester.

The memory 512 may include one or more image processing modules 518 that, when executed, may cause the processor 506 to extract optical data from a data stream received from the endoscopic vein harvesting system 110 and to provide the optical data and other data to the display 114. The image processing modules 518 may also store the optical data together with other data from the data stream as endoscopic procedure data 522 within the memory device 512. In some implementations, the image processing modules 518 may selectively process the optical data to enhance the visual data presented on the display 114. In an example, the image processing modules 518 may utilize edge detection algorithms, contrast enhancement algorithms, and other algorithms to amplify contrast data within the optical data to assist the harvester in visualizing the procedure.

The memory 512 may include one or more cauterizer control modules 520 that, when executed, may cause the processor 506 to communicate with the cauterizer 206 of the components 526 of the endoscopic vein harvesting system 110. In some implementations, the cauterizer control modules 520 may monitor sensor data associated with the cauterizer 206 and may shut down the cauterizer 206 when sensor data exceeds a threshold. For example, if a temperature of the cauterizer exceeds a safe temperature, the cauterizer control module 520 may cause the processor 506 to present a warning on the display 114 and to shut off power to the cauterizer.

The memory 512 may include other modules 524 that, when executed, may cause the processor 506 to perform other operations. In an example, the other modules 524 may include printer drivers that may be used by the processor 506 to send data to a printer device. In another example, the other modules 524 may include operating system instructions that may be executed by the processor 506 to enable ordinary functionality of the computing device 112 including communicating with the display 114 and receiving data from the one or more input devices 502. The other modules 524 may include instructions for other functions as well. For example, the other modules 524 may enable updates of instructions stored in the memory devices 512.

In some implementations, the computing device 112 may store data 525, which may include images, sensor readings, and other data determined from the endoscopic vein harvesting system 110. The data 525 may be stored and subsequently communicated by the processor 506 executing the one or more patient modules 514 to one or more other systems via the network 510, such as to the data sources 528 for storage. Other implementations are also possible.

In conjunction with the systems, methods, and devices described above with respect to FIGS. 1A-5, a system is described in which components 526 attached to an endoscope 116 are provided with visual indicators 134 that can be seen on a display 114 of the computing device 112 that is coupled to the endoscopic vein harvesting system 110. The visual indicators 134 may be formed from one or more lines or other shapes and may be formed from a color that provides a high contrast relative to the background biological material. In some implementations, the visual indicator 134 may be formed on a component 526 and may have a fluorescent green color (or another color) that does not naturally appear in the human body and that provides a high contrast relative to blood and other biological material.

The visual indicators 134 may be provided on the tip 132 of a dissection cap 118, on an edge of a manipulation device 202, on one or more tips of a bisector device 204, or any combination thereof. The visual indicators 134 may be provided on any component of the endoscopic harvesting system 110 for which the precise position is difficult to discern relative to the surrounding biological material. The visual indicator 134 may be viewed on the display 114 and may be used by the harvester to determine the position of the component 526 relative to the vein 104 to be harvested.

Although the present invention has been described with reference to preferred embodiments, workers skilled in the art will recognize that changes may be made in form and detail without departing from the scope of the invention.

What is claimed is:

1. An endoscopic vein harvesting system comprising:
an endoscope including a proximal end having one or more of a collar or a handle and including a distal end;
a component formed from a transparent material and configured to couple to the distal end of the endoscope; and
one or more visual indicators formed on or within a selected portion of the component.

2. The endoscopic vein harvesting system of claim 1, wherein the one or more visual indicators comprise a color selected to have a high contrast relative to biological material corresponding to a vein to be harvested.

3. The endoscopic vein harvesting system of claim 2, wherein the color comprises a fluorescent green color.

4. The endoscopic vein harvesting system of claim 1, wherein the one or more visual indicators comprise one or more of a line, a crosshair, a triangle, an arrow, or a dot.

5. The endoscopic vein harvesting system of claim 1, wherein:
the component comprises a dissection cap including a tip; and
the one or more visual indicators are formed on or within the tip.

6. The endoscopic vein harvesting system of claim 1, wherein:
the component comprises a bisector including a first tip and a second tip on either side of an opening; and
the one or more visual indicators are formed on the first tip and the second tip.

7. The endoscopic vein harvesting system of claim 1, wherein:
the component comprises a manipulation device including a surface configured to engage a vein; and
the one or more visual indicators are formed on the surface to provide a visible guide to facilitate alignment of the manipulation device to the vein.

8. The endoscopic vein harvesting system of claim 1, further comprising:
a display; and
a computing device coupled to the endoscope and the display, the computing device configured to receive optical data from the endoscope that includes image data captured through a distal end of the endoscope, the optical data including images of one or more of a vein, a carbon dioxide tunnel around the vein, or the surrounding biological material and the one or more visual indicators on the component, the computing device configured to provide at least a portion of the optical data that includes the one or more visual indicators to the display.

9. An endoscopic vein harvesting system comprising:
an endoscope including a proximal end including one or more of a collar or a handle and including a distal end to be inserted through an incision into a patient;
a component formed from a transparent material and configured to couple to the distal end of the endoscope prior to insertion, the component including one or more visual indicators formed on or within a selected portion of the component to facilitate positioning of the component relative to a vein to be harvested; and
a computing system coupled to the endoscope and configured to receive optical data corresponding to a view through the distal end of the endoscope, the optical data including images of the vein, surrounding biological material, and the one or more visual indicators, the computing system including a display and configured to present at least a portion of the optical data including the one or more visual indicators to the display.

10. The endoscopic vein harvesting system of claim 9, wherein the one or more visual indicators comprise a color selected to have a high contrast relative to the biological material and the vein to be harvested.

11. The endoscopic vein harvesting system of claim 10, wherein the color comprises a fluorescent green color.

12. The endoscopic vein harvesting system of claim 9, wherein the one or more visual indicators comprise one or more of a line, a crosshair, a triangle, an arrow, or a dot.

13. The endoscopic vein harvesting system of claim 9, wherein:
the component comprises a dissection cap including a tip; and
the one or more visual indicators are formed on or within the tip.

14. The endoscopic vein harvesting system of claim 9, wherein:
the component comprises a bisector including a first tip and a second tip on either side of an opening; and
the one or more visual indicators are formed on the first tip and the second tip.

15. The endoscopic vein harvesting system of claim 9, wherein:
the component comprises a manipulation device including a surface to engage the vein; and
the one or more visual indicators are formed on the surface to provide a visible guide to align the component to the vein.

16. An endoscopic vein harvesting system comprising:
an endoscope including a proximal end having one or more of a collar or a handle and including a distal end;
a component formed from a transparent material and configured to couple to the distal end of the endoscope; and
one or more visual indicators formed on or within a selected portion of the component and comprising a color selected to have a high contrast relative to biological material corresponding to a vein to be harvested.

17. The endoscopic vein harvesting system of claim 16, wherein the color comprises a fluorescent green color.

18. The endoscopic vein harvesting system of claim 16, wherein the one or more visual indicators comprise one or more of a line, a crosshair, a triangle, an arrow, or a dot.

19. The endoscopic vein harvesting system of claim 1, wherein the component comprises one or more of:
a dissection cap including a tip;
a bisector including a first tip and a second tip on either side of an opening; or
a manipulation device including a surface configured to engage a vein; and
wherein the one or more visual indicators are formed on one or more of the dissection cap, the tip, the first tip, the second tip, or the surface to provide a visible guide to facilitate alignment of the manipulation device to the vein.

20. The endoscopic vein harvesting system of claim 16, further comprising:
a display; and
a computing device coupled to the endoscope and the display, the computing device configured to receive optical data from the endoscope that includes image data captured through a distal end of the endoscope, the optical data including images of one or more of a vein, a carbon dioxide tunnel around the vein, or the surrounding biological material and the one or more visual indicators on the component, the computing device configured to provide at least a portion of the optical data that includes the one or more visual indicators to the display.
